(19) United States
(12) Patent Application Publication
(10) Pub. No.: US 2024/0041986 A1
(43) Pub. Date: Feb. 8, 2024

(54) METHODS AND COMPOSITIONS FOR THE TREATMENT AND PREVENTION OF TYPE 1 DIABETES

(71) Applicants: Dr. Mary Morris & Associates, LLC, Onalaska, WI (US); Saint Louis University, St. Louis, MO (US)

(72) Inventors: Mary S. Morris, Onalaska, WI (US); Richard J. DiPaolo, Saint Louis, MO (US)

(73) Assignees: Dr. Mary Morris & Associates, LLC, Onalaska, WI (US); Saint Louis University, St. Louis, MO (US)

(21) Appl. No.: 18/028,634

(22) PCT Filed: Oct. 5, 2021
(86) PCT No.: PCT/US2021/053537

§ 371 Date: Mar. 27, 2023

Related U.S. Application Data
(60) Provisional application No. 63/089,122, filed on Oct. 8, 2020.

(51) Int. Cl. A61K 38/28; A61K 47/02; A61K 47/10; A61P 3/10

(52) U.S. Cl. CPC A61K 38/28; A61K 47/02; A61K 47/10; A61P 3/10

(57) ABSTRACT

Methods of attenuating an antigenic response in a mammal to one or more Type 1 diabetes related-antigens are provided. The method may result in delaying the onset of decreased pancreatic beta cell function in the mammal and/or at least delaying a reduction in serum C-peptide levels in the mammal The method comprises sublingually administering an effective amount of an insulin-related peptide to the mammal and commonly makes use of a sublingual formulation of an insulin-related peptide that includes an aqueous pharmaceutically acceptable carrier, e.g., an aqueous carrier which comprises at least about 30 vol. % glycerin.

### CROSS-REFERENCE TO RELATED APPLICATIONS

The present application claims the benefit of and priority to U.S. Provisional Application No. 63/089,122, filed Oct. 8, 2020, the contents of which is incorporated herein by reference in their entirety.

### SEQUENCE LISTING

The instant application contains a Sequence Listing which has been submitted electronically in ASCII format and is hereby incorporated by reference in its entirety. Said ASCII copy, created on Oct. 4, 2021, is named 120177-0109_SL.txt and is 15,417 bytes in size.

### BACKGROUND

Type 1 diabetes (T1D; also known as “autoimmune diabetes,” and formerly known as “insulin-dependent diabetes,” or “juvenile-onset diabetes”) is a chronic disease that results from an autoimmune-mediated destruction of pancreatic β-cells with consequent loss of insulin production, which manifests clinically as hyperglycemia, and accounts for 5-10% of all cases of diabetes. The age of symptomatic onset is usually during childhood or adolescence; however, symptoms can develop much later in life. Although the etiology of T1D is not completely understood, the pathogenesis is thought to involve T cell-mediated destruction of pancreatic β-cells. There is no known cure for T1D, and patients must rely on daily insulin therapy to compensate for impaired β-cell function. Insulin treatments typically involve either multiple daily insulin injection therapy or continuous subcutaneous insulin infusion. Without insulin, these patients develop serious complications such as ketoacidosis, retinopathy, nephropathy, vasculopathy, and neuropathy. Because subcutaneous delivery of insulin requires strict, self-regimentation, compliance is often a serious problem. Moreover, the act of parenteral insulin administration can be traumatic for juveniles. Treatment of T1D with exogenous insulin can result in exogenous insulin antibody syndrome, also known as Hirata's disease, which leads to hypoglycemia. Presently, there are no known effective oral or sublingual insulin therapies. Compliance concerns coupled with serious morbidity and an increasing incidence of T1D worldwide, underscore the need to develop effective therapies for T1D prevention and/or treatment.

### SUMMARY

The present technology relates generally to methods for attenuating an antigenic response in a mammal to one or more Type 1 diabetes related-antigens. Very often, the method comprises attenuating the antigenic response in the mammal to an insulin peptide and, optionally, to one or more other Type 1 diabetes related-antigens. The method comprises sublingually administering an effective amount of an insulin-related peptide to the mammal. The method may result in inhibiting development of anti-insulin antibodies (IA) in the mammal after sublingual administration of the insulin-related peptide as compared to a control mammalian subject.

In one embodiment, a method for delaying the onset of decreased pancreatic beta cell function in a mammal is provided. The method comprises sublingually administering an insulin-related peptide to the mammal in an amount effective to conserve serum C-peptide levels in the mammal.

Another embodiment is directed to a method for conserving pancreatic beta cell function in a mammal. The method comprises sublingually administering an insulin-related peptide to the mammal in an amount effective to at least delay a reduction in serum C-peptide levels in the mammal.

In another embodiment, a method for attenuating an antigenic response in a mammal to one or more Type 1 diabetes related-antigens is provided. The method includes sublingually administering an insulin-related peptide to the mammal in an amount of effective to inhibit development of antibodies to at least one Type 1 diabetes related-antigen. For example, sublingually administering the insulin-related peptide to the mammal may inhibit development of antibodies to one or more Type 1 diabetes related-antigens, such as an insulin, glutamic acid decarboxylase 65 (GAD65), insulinoma-associated protein 2 (IA-2), zinc transporter-8 (ZnT8), and islet amyloid polypeptide (IAPP).

In another embodiment, a method for delaying the onset of reduced serum C-peptide levels in a mammal is provided. The method comprises sublingually administering an effective amount of an insulin-related peptide to the mammal.

The present methods typically use a sublingual formulation of an insulin-related peptide. In addition to containing the insulin-related peptide, the sublingual formulation commonly includes an aqueous pharmaceutically acceptable carrier, e.g., an aqueous carrier which comprises at least about 30 vol. % glycerin. Examples of suitable insulin-related peptides are peptides which include a first amino acid sequence comprising an insulin beta chain 7-26 peptide sequence (SEQ ID NO: 9) or a variant thereof having one or more amino acid substitutions; and a second amino acid sequence comprising an insulin alpha chain 6-20 peptide sequence (SEQ ID NO: 4) or a variant thereof having one or more amino acid substitutions. The sublingual formulation of the insulin-related peptide may be capable of significantly reducing the incidence and delaying the onset of T1D in an art-accepted mouse model of the disease (the non-obese diabetic (NOD) mouse).

Another embodiment is directed to a method of attenuating an antigenic response in a mammal to at least one Type 1 diabetes related-antigen. The method includes sublingually administering an effective amount of an insulin-related peptide to the mammal. Examples of suitable insulin-related peptides are peptides which include a first amino acid sequence comprising an insulin beta chain 7-26 peptide sequence (SEQ ID NO: 9) or a variant thereof having one or more amino acid substitutions; and a second amino acid sequence comprising an insulin alpha chain 6-20 peptide sequence (SEQ ID NO: 4) or a variant thereof having one or more amino acid substitutions. After sublingual administration of the insulin-related peptide, the subject may display reduced levels of autoantibodies, such as islet cell antibodies (ICA), glutamic acid decarboxylase-65 (GAD-antibodies, insulin autoantibodies (IAA), exogenous insulin associated antibodies (EIA), insulinoma-associated protein 2A (IA-2A) autoantibodies, insulinoma-associated protein 2β (IA-2β) autoantibodies, and/or zinc transporter 8 (ZnT8) autoantibodies).

### BRIEF DESCRIPTION OF THE DRAWINGS

FIGS. 1A and 1B are graphs showing the incidence (%) and time to onset (Weeks) of Type 1 diabetes in control NOD mice and in NOD mice sublingually treated five (5) times per week with 87 μg Humulin® insulin beginning at 6 weeks of age (FIG. 1A) and at weeks of age (FIG. 1B).

FIG. 2 is a graph showing anti-insulin antibodies in serum collected from control NOD mice and in NOD mice at 14 weeks of age after sublingual treatment five (5) times per week with 87 μg Humulin® insulin beginning at 6 weeks of age (as determined by ELISA). **p=0.0011.

FIG. 3 is a graph showing the incidence (% Diabetic) and time to onset (Weeks) of Type 1 diabetes in a separate experiment that included control NOD mice and in NOD mice sublingually treated five (5) times per week with 87 μg Humulin® insulin (Humulin SLIT) beginning at 5 weeks of age.

FIG. 4 is a graph showing anti-insulin antibodies in serum collected from control NOD mice and in NOD mice at 19 weeks of age after sublingual treatment five (5) times per week with 87 μg Humulin® insulin beginning at 5 weeks of age (as determined by ELISA). **p=0.0001.

FIG. 5 provides graphs showing serum C-peptide levels at 6 weeks and at 19 weeks of age in control NOD mice (Control Group) and in NOD mice sublingually treated five (5) times per week with 87 μg Humulin® insulin (Humulin SLIT) beginning at 5 weeks of age. **p=0.006 for control group, P=0.2 for the Humulin SLIT group

FIG. 6 is a chart showing the combined results of control and Humulin® treated mice shown in FIG. 1A and FIG. 3 for the incidence (% Diabetic) and time to onset (Weeks) of Type 1 diabetes in control NOD mice and in NOD mice sublingually treated five (5) times per week with 87 μg Humulin® insulin (Humulin SLIT) beginning at 5 or 6 weeks of age.

FIG. 7A is a graph showing the incidence (Percent Diabetic) and time to onset (Weeks) of Type 1 diabetes in control NOD mice and in NOD mice sublingually treated five (5) times per week with a composition comprising 52 μg Humulin® insulin, 10 preproinsulin synthetic peptide, and 10 μg insulin beta chain 9-23 synthetic peptide (Peptides SLIT) beginning at 5 weeks of age.

FIG. 7B is a graph showing anti-insulin antibodies in the serum of control NOD mice and in NOD mice at 19 weeks of age sublingually treated five (5) times per week with a composition comprising 52 μg Humulin® insulin, 10 μg preproinsulin synthetic peptide, and 10 μg insulin beta chain 9-23 synthetic peptide (Humulin+Peptides) beginning at 5 weeks of age as determined by ELISA. *p=0.0496.

### DETAILED DESCRIPTION

### I. Definitions

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include both singular and plural referents unless the content clearly dictates otherwise. For example, reference to “a cell” includes a combination of two or more cells, and the like.

As used herein, the “administration” of an agent, drug, or peptide to a subject refers to sublingual administration of the compositions of the present technology to the subject.

As used herein, “attenuating” or “attenuated” antigenic response means a decrease in synthesis of antibodies that are associated with T1D. Such antibodies include autoantibodies associated with T1D, such as insulin autoantibodies (IAA), islet cell antibodies (ICA), 65 kDa glutamic acid decarboxylase (GAD-65), insulinoma-associated protein 2A or 2β (IA-2A, IA-2β), or zinc transporter 8 (ZnT8), and antibodies against exogenous insulin.

As used herein, a “conservative amino acid substitution” is one that does not substantially change the structural and functional characteristics of the parent sequence (e.g., a replacement amino acid should not tend to break a helix that occurs in the parent sequence, or disrupt other types of secondary structure that characterize the parent sequence or are necessary for its functionality).

As used herein, the term “effective amount” refers to a quantity sufficient to achieve a desired therapeutic and/or prophylactic effect, e.g., an amount which results in partial or full amelioration of one or more symptoms of Type 1 diabetes. In the context of therapeutic or prophylactic applications, in some embodiments, the amount of a composition administered to the subject will depend on the type, degree, and severity of the disease and on the characteristics of the individual, such as general health, age, sex, body weight, and tolerance to drugs. The skilled artisan will be able to determine appropriate dosages depending on these and other factors. The compositions can also be administered in combination with one or more additional therapeutic compounds. For example, in the methods described herein, insulin-related peptides of the present technology, such as Humulin® or a variant thereof having one or more conservative amino acid substitutions, may be administered to a subject having one or more signs, symptoms, or risk factors of Type 1 diabetes, including, but not limited to, hyperglycemia, hypoinsulinemia, reduced serum C-peptide levels, elevated A1C levels, presence of T1D-associated autoantibodies or exogenous insulin associated antibodies, excessive excretion of urine (polyuria), thirst (polydipsia), constant hunger (polyphagia), weight loss, vision changes, fatigue, mental confusion, nausea, vomiting, ketoacidosis, retinopathy, nephropathy, vasculopathy, and neuropathy. The insulin-related peptides may also be administered to a disease-free subjects genetically predisposed to the development of T1D (e.g., first-degree relatives of patients with Type 1 diabetes, where the relatives have been determined to be genetically predisposed to the development of Type 1 diabetes). For example, a “therapeutically effective amount” of the insulin-related peptides includes levels at which the presence, frequency, or severity of one or more signs, symptoms, or risk factors of Type 1 diabetes are, at a minimum, ameliorated. A therapeutically effective amount may reduce or ameliorate the physiological effects of Type 1 diabetes, and/or the risk factors of Type 1 diabetes, and/or the likelihood of developing Type 1 diabetes. A therapeutically effective amount can be given in one or more administrations.

As used herein, the term “insulin-related peptide” refers to a peptide comprising a first amino acid sequence comprising an insulin beta chain (B-chain) or biologically active fragment thereof or a variant of either of these having one or more amino acid substitutions and/or a second amino acid sequence comprising an insulin alpha chain (A-chain) or biologically active fragment thereof or a variant of either of these having one or more amino acid substitutions. In some embodiments, the insulin-related peptide comprises an amino acid sequence comprising an insulin beta chain 7-26 peptide sequence (SEQ ID NO: 9) or a variant thereof having one or more amino acid substitutions. In some embodiments, the insulin-related peptide comprises an amino acid sequence comprising an insulin alpha chain 6-20 peptide sequence (SEQ ID NO: 4) or a variant thereof having one or more amino acid substitutions. In some embodiments, the insulin-related peptide comprises a first amino acid sequence comprising an insulin beta chain 7-26 peptide sequence (SEQ ID NO: 9) or a variant thereof having one or more amino acid substitutions and a second amino acid sequence comprising an insulin alpha chain 6-20 peptide sequence (SEQ ID NO: 4) or a variant thereof having one or more amino acid substitutions. In some embodiments, the insulin-related peptides comprise a first amino acid sequence comprising a human insulin beta chain 7-26 peptide sequence (SEQ ID NO: 9) or a variant thereof having one or more amino acid substitutions and a second amino acid sequence comprising a human insulin alpha chain 6-20 peptide sequence (SEQ ID NO: 4) or a variant thereof having one or more amino acid substitutions. In some embodiments, “insulin-related peptides” include larger fragments of the insulin beta and insulin alpha chains. For example, in some embodiments, the insulin-related peptide may include an insulin beta chain 6-26 peptide sequence (SEQ ID NO: 3), an insulin beta chain 3-26 peptide sequence (SEQ ID NO: 7), an insulin beta chain 4-27 peptide sequence (SEQ ID NO: 8), an insulin beta chain sequence (SEQ ID NO: 1), an insulin alpha chain 1-20 peptide sequence (SEQ ID NO: 5), an insulin alpha chain 4-20 peptide sequence (SEQ ID NO: 6), or an insulin alpha chain sequence (SEQ ID NO: 2), or variants thereof. The insulin-related peptide may be of human origin or of any mammalian species. In some embodiments, the insulin-related peptide is a recombinant human insulin-related peptide, such as Humulin® or a variant thereof having one or more conservative amino acid substitutions. In some embodiments, the insulin-related peptide comprises one or more of insulin, proinsulin, and preproinsulin.

As used herein, the term “Type 1 diabetes” or “T1D,” refers to a disorder characterized by insulin deficiency due to pancreatic β-cell loss that leads to hyperglycemia. T1D can be diagnosed using a variety of diagnostic tests as described below. These include, but are not limited to, (1) glycated hemoglobin A1C (HbA1C) test (HbA1C level≥6.5%), (2) oral glucose tolerance test (OGTT; post-load plasma glucose level≥200 mg/dL), (3) random blood glucose test (glucose level≥200 mg/dL at any time of day combined with symptoms of diabetes), (4) fasting plasma glucose (FPG) test (fasting blood sugar≥126 mg/dL), (5) C-peptide level of less than 0.2 nmol/L.

“Treating” or “treatment” as used herein covers the treatment of Type 1 diabetes and/or its signs or symptoms in a subject, such as a human, and includes: (i) inhibiting Type 1 diabetes, i.e., arresting its development; (ii) relieving Type 1 diabetes, i.e., causing regression of the disorder; (iii) slowing the progression of Type 1 diabetes; and/or (iv) inhibiting, relieving, or slowing progression of one or more signs or symptoms of Type 1 diabetes, including, but not limited to, hyperglycemia, hypoinsulinemia, reduced serum C-peptide levels, elevated A1C levels, presence of T1D-associated autoantibodies or exogenous insulin associated antibodies, polyuria, polydipsia, polyphagia, weight loss, vision changes, fatigue, mental confusion, nausea, vomiting, and ketoacidosis.

As used herein, “preventing” or “prevention” of a disorder or condition refers to a compound that reduces the occurrence or likelihood of the disorder or condition in the treated sample relative to an untreated control sample, or delays the onset of one or more signs or symptoms of the disorder or condition relative to the untreated control sample, including, but not limited to, hyperglycemia, hypoinsulinemia, reduced serum C-peptide levels, elevated A1C levels, presence of T1D-associated autoantibodies, exogenous insulin associated antibodies (EIA), polyuria, polydipsia, polyphagia, weight loss, vision changes, fatigue, mental confusion, nausea, vomiting, and ketoacidosis. As used herein, preventing Type 1 diabetes refers to preventing or delaying the onset of Type 1 diabetes. As used herein, prevention of Type 1 diabetes also includes preventing a recurrence of one or more signs or symptoms of Type 1 diabetes.

It is also to be appreciated that the various modes of treatment or prevention of medical conditions as described herein are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

As used herein, the terms “subject” and “patient” are used interchangeably.

### II. General

The present technology relates to the surprising discovery of a sublingual formulation of insulin-related peptide that is capable of significantly reducing the incidence and delaying the onset of T1D in an art-accepted mouse model of the disease (the non-obese diabetic (NOD) mouse). Effective sublingual insulin treatment for T1D is a highly unmet need. The methods and compositions of the present technology therefore provide a desirable route of administration that is efficacious as a T1D therapeutic and may improve patient compliance.

### III. Insulin-Related Peptides

Insulin hormone is a 51-amino acid protein that is secreted by pancreatic β-cells in the Islets of Langerhans. Insulin is first synthesized as preproinsulin in the rough endoplasmic reticulum of the pancreatic β-cells. After the signal peptide in the preprohormone is removed by proteolytic cleavage, a proinsulin molecule composed of an alpha chain (or A-chain) peptide with 21 amino acids, a beta chain (or B-chain) peptide with 30 amino acids, and an intervening C chain peptide (C-peptide) is produced. Subsequent processing of proinsulin in the Golgi complex produces biologically active insulin by removing the C-peptide and linking the alpha and beta chains through two disulfide bonds at cysteine residues. A third disulfide bond connects two cysteine residues within the alpha chain. Insulin and C-peptide are secreted simultaneously in equimolar amounts in response to various stimuli, such as glucose.

As illustrated by Table 1, the A-chain and B-chain amino acid sequences of insulin are highly conserved among vertebrates. In addition, the positions of the three disulfide bonds are also the same for most species. These highly conserved characteristics lead to a three dimensional conformation of insulin that is very similar across species. For this reason, insulin from one species is often biologically active and has similar physiological effects in other species. Table 1 discloses SEQ ID NOS 10-24, respectively, in order of appearance.

TABLE 1
Insulin sequences from certain species.

| Vertebrate | | | | | |
|---|---|---|---|---|---|
| HUMAN | FVNQHLCGSHLVEAL | RR | EAEDLQVGQVELGGGP | KR | GIVEQCCTSIC |
| | YLVCGERGFFYTPKT | | GAGSLQPLALEGSLQ | | SLYQLENYCN |
| GREAT APES | FVNQHLCGSHLVEAL | RR | EAEDLQVGQVELGGGP | KR | GIVEQCCTSIC |
| | YLVCGERGFFYTPKT | | GAGSLQPLALEGSLQ | | SLYQLENYCN |
| MACAQUE | FVNQHLCGSHLVEAL | RR | EAEDPQVGQVELGGGP | KR | GIVEQCCTSIC |
| (CYNOMOLGUS) | YLVCGERGFFYTPKT | | GAGSLQPLALEGSLQ | | SLYQLENYCN |
| RABBIT | FVNQHLCGSHLVEAL | RR | EVEELQVGQAELGGGP | KR | GIVEQCCTSIC |
| | YLVCGERGFFYTPKS | | GAGGLQPLALELALQ | | SLYQLENYCN |
| CANINE | FVNQHLCGSHLVEAL | RR | EVEDLQVRDVELAGAP | KR | GIVEQCCTSIC |
| | YLVCGERGFFYTPKA | | GEGGLQPLALEGALQ | | SLYQLENYCN |
| EQUINE | FVNQHLCGSHLVEAL | XX | EAEDPQVGEVELGGGP | XX | GIVEQCCTGIC |
| | YLVCGERGFFYTPKA | | GLGGLQPLALAGPQQ | | SLYQLENYCN |
| PORCINE | FVNQHLCGSHLVEAL | RR | EAENPQAGAVELGGGL | KR | GIVEQCCTSIC |
| | YLVCGERGFFYTPKA | | GG--LQALALEGPPQ | | SLYQLENYCN |
| HAMSTER | FVNQHLCGSHLVEAL | RR | GVEDPQVAQLELGGGP | KR | GIVDQCCTSIC |
| | YLVCGERGFFYTPKS | | GADDLQTLALEVAQQ | | SLYQLENYCN |
| RAT II | FVKQHLCGSHLVEAL | RR | EVEDPQVAQLELGGGP | KR | GIVDQCCTSIC |
| | YLVCGERGFFYTPMS | | GAGDLQTLALEVARQ | | SLYQLENYCN |
| MOUSE II | FVKQHLCGSHLVEAL | RR | EVEDPQVAQLELGGGP | KR | GIVDQCCTSIC |
| | YLVCGERGFFYTPMS | | GAGDLQTLALEVAQQ | | SLYQLENYCN |
| FELINE | FVNQHLCGSHLVEAL | RR | EAEDLQGKDAELGEAP | KR | GIVEQCCASVC |
| | YLVCGERGFFYTPKA | | GAGGLQPLALEAPLQ | | SLYQLEHYCN |
| RAT I | FVKQHLCGPHLVEAL | RR | EVEDPQVPQLELGGGP | KR | GIVDQCCTSIC |
| | YLVCGERGFFYTPKS | | EAGDLQTLALEVARQ | | SLYQLENYCN |
| HOUSE I | FVKQHLCGPHLVEAL | RR | EVEDPQVEQLELGGSP | KR | GIVDQCCTSIC |
| | YLVCGERGFFYTPKS | | --GDLQTLALEVARQ | | SLYQLENYCN |
| BOVINE | FVNQHLCGSHLVEAL | RR | EVEGPQVGALELAGGP | KR | GIVEQCCASVC |
| | YLVCGERGFFYTPKA | | G-----AGGLEGPPQ | | SLYQLENYCN |
| OVINE | FVNQHLCGSHLVEAL | RR | EVEGPQVGALELAGGP | KR | GIVEQCCAGVC |
| | YLVCGERGFFYTPKA | | G-----AGGLEGPPQ | | SLYQLENYCN |
| | Insulin B-chain | | C-peptide | | Insulin A-chain |

The insulin-related peptides of the present technology, which are formulated for sublingual administration, include a peptide comprising an amino acid sequence comprising an insulin beta chain (B-chain) or a biologically active fragment thereof or a variant of either of these having one or more amino acid substitutions, and may include any one or more of the B-chain sequences as shown in Table 1. In some embodiments, the insulin-related peptides of the present technology, which are formulated for sublingual administration, include a peptide comprising an amino acid sequence comprising an insulin alpha chain (A-chain) or a biologically active fragment thereof or a variant of either of these having one or more amino acid substitutions, and may include any one or more of the A-chain sequences as shown in Table 1. In some embodiments, the insulin-related peptides of the present technology, which are formulated for sublingual administration, include a peptide comprising a first amino acid sequence comprising an insulin beta chain (B-chain) or a biologically active fragment thereof or a variant of either of these having one or more amino acid substitutions and a second amino acid sequence comprising an insulin alpha chain (A-chain) or a biologically active fragment thereof or a variant of either of these having one or more amino acid substitutions, and may include any one or more of the B-chain and A-chain sequences as shown in Table 1. In some embodiments, the insulin-related peptides of the present technology include an insulin beta chain 7-26 peptide sequence (SEQ ID NO: 9) or a variant thereof having one or more amino acid substitutions and a second amino acid sequence comprising an insulin alpha chain 6-20 peptide sequence (SEQ ID NO: 4) or a variant thereof having one or more amino acid substitutions. In some embodiments, the insulin-related peptides of the present technology include larger fragments of the insulin beta and insulin alpha chains. For example, in some embodiments, the insulin-related peptide may include an insulin beta chain 6-26 peptide sequence (SEQ ID NO: 3), an insulin beta chain 3-26 peptide sequence (SEQ ID NO: 7), an insulin beta chain 4-27 peptide sequence (SEQ ID NO: 8), an insulin beta chain sequence (SEQ ID NO: 1), an insulin alpha chain 1-20 peptide sequence (SEQ ID NO: 5), an insulin alpha chain 4-20 peptide sequence (SEQ ID NO: 6), or an insulin alpha chain sequence (SEQ ID NO: 2). The insulin-related peptide may be of human origin or of any mammalian species. For example, the insulin-related peptides of the present technology may include any one or more of the insulin A-chains or B-chains shown in Table 1. In some embodiments, the insulin-related peptide is a recombinant human insulin-related peptide, such as Humulin® or a variant thereof having one or more conservative amino acid substitutions. In some embodiments, the insulin-related peptide comprises one or more of insulin, proinsulin, and preproinsulin. In some embodiments, the insulin-related peptide is a fast-acting, intermediate-acting, or long-acting insulin analog.

In addition to the insulin-related peptide sequences provided in Table 1, exemplary, non-limiting insulin-related peptides of the present technology are also provided in Table 2.

TABLE 2
Exemplary insulin-related peptides.
Insulin-related peptide alpha
chain peptides and fragments

| GlyIleValGluGlnCysCysThrSerIleCys | SEQ ID NO: |
|---|---|
| SerLeuTyrGlnLeuGluAsnTyrCysAsn | 2 |
| CysCysThrSerIleCysSerLeuTyrGlnLeu | SEQ ID NO: |
| GluAsnTyrCys | 4 |
| GlyIleValGluGlnCysCysThrSerIleCys | SEQ ID NO: |
| SerLeuTyrGlnLeuGluAsnTyrCys | 5 |
| GluGlnCysCysThrSerIleCysSerLeuTyr | SEQ ID NO: |
| GlnLeuGluAsnTyrCys | 6 |

Insulin-related peptide beta
chain peptides and fragments

| | |
|---|---|
| PheValAsnGlnHisLeuCysGlySerHisLeu | SEQ ID NO: |
| ValGluAlaLeuTyrLeuValCysGlyGluArg | 1 |
| GlyPhePheTyrThrProLysThr | |
| LeuCysGlySerHisLeuValGluAlaLeuTyr | SEQ ID NO: |
| LeuValCysGlyGluArgGlyPhePheTyr | 3 |
| AsnGlnHisLeuCysGlySerHisLeuValGlu | SEQ ID NO: |
| AlaLeuTyrLeuValCysGlyGluArgGlyPhe | 7 |
| PheTyr | |
| GlnHisLeuCysGlySerHisLeuValGluAla | SEQ ID NO: |
| LeuTyrLeuValCysGlyGluArgGlyPhePhe | 8 |
| TyrThr | |
| CysGlySerHisLeuValGluAlaLeuTyrLeu | SEQ ID NO: |
| ValCysGlyGluArgGlyPhePheTyr | 9 |

Suitable substitution variants of the peptides listed herein include conservative amino acid substitutions. Amino acids may be grouped according to their physicochemical characteristics as follows:
(a) Non-polar amino acids: Ala(A) Ser(S) Thr(T) Pro(P) Gly(G) Cys (C);
(b) Acidic amino acids: Asn(N) Asp(D) Glu(E) Gln(Q);
(c) Basic amino acids: His(H) Arg(R) Lys(K);
(d) Hydrophobic amino acids: Met(M) Leu(L) Ile(I) Val(V); and
(e) Aromatic amino acids: Phe(F) Tyr(Y) Trp(W).

Substitutions of an amino acid in a peptide by another amino acid in the same group are referred to as a conservative substitution and may preserve the physicochemical characteristics of the original peptide. In other embodiments, variants of the peptides described herein may include one or more of the following substitutions:
Asn substituted by Lys, His, or Gly
Glu substituted by Asp
Ile substituted by Ala, Gly, Leu, or Val
Lys substituted by Met
Ser substituted by Thr, Gly, Ala, or Pro
Thr substituted by Ala, Ser, Gly, or Val.

The peptides may be synthesized by any of the methods well known in the art. Suitable methods for chemically synthesizing the protein include, for example, those described by Stuart and Young in Solid Phase Peptide Synthesis, Second Edition, Pierce Chemical Company (1984), and in Methods Enzymol., 289, Academic Press, Inc., New York (1997).

### IV. Type 1 Diabetes

Type 1 diabetes (T1D), also known as “autoimmune diabetes,” (previously known as “insulin-dependent diabetes,” or “juvenile-onset diabetes”) is a chronic disease characterized by insulin deficiency due to pancreatic β-cell loss that leads to hyperglycemia. The age of symptomatic onset is usually during childhood or adolescence; however, symptoms can sometimes develop much later. Although the etiology of T1D is not completely understood, the pathogenesis of the disease is thought to involve T cell-mediated destruction of β-cells. A cure is not available, and patients depend on lifelong insulin injections. Although intensive glycemic control has reduced the incidence of microvascular and macrovascular complications, the majority of patients with T1D are still developing these complications.

### A. Clinical Manifestations

The clinical signs and symptoms of T1D include hyperglycemia, hypoinsulinemia, reduced serum C-peptide levels, elevated A1C levels, presence of T1D-associated autoantibodies, excessive excretion of urine (polyuria), thirst (polydipsia), constant hunger (polyphagia), weight loss, vision changes, fatigue, mental confusion, nausea, vomiting, and ketoacidosis. Chronic symptoms of T1D include retinopathy, nephropathy, vasculopathy, and neuropathy.

### B. Diagnosis

T1D in humans is diagnosed by a combination of symptoms and the results of certain blood tests. In a fasting plasma glucose (FPG) test, diabetes is diagnosed if a fasting blood sugar level is 126 mg/dL or higher. In an oral glucose tolerance test (OGTT), diabetes is diagnosed if the 2-hour post-load plasma glucose level is 200 mg/dL or higher. In a random blood glucose test, a blood glucose level of 200 mg/dL or greater at any time of day combined with symptoms of diabetes is sufficient to make the diagnosis. In a hemoglobin A1C (HbA1C; glycohemoglobin) test, which measures the average glucose level over the prior two to three months, diabetes is diagnosed if the HbA1C level is 6.5% or higher. If elevated values are detected in asymptomatic people, repeat testing, preferably with the same test, is recommended as soon as practicable on a subsequent day to confirm the diagnosis. Endogenous insulin production can be assessed by measuring serum C-peptide either in the fasting state or after a stimulus, most commonly intravenously administered glucagon. C-peptide can also be measured in urine. The normal range for fasting serum C-peptide levels in humans is 0.26 to 1.27 nmol/L. A C-peptide level of less than 0.2 nmol/L is associated with a diagnosis of T1D in humans.

Progression to T1D is typically preceded by a prodrome of anti-islet autoantibody expression. Biomarkers of T1D-associated autoimmunity that may be found months to years before symptom onset include a number of T1D-associated autoantibodies such as insulin autoantibodies (IAA), islet cell antibodies (ICA), 65 kDa glutamic acid decarboxylase (GAD-65), insulinoma-associated protein 2A or 2β (IA-2A, IA-2β), and zinc transporter 8 (ZnT8), which are proteins associated with secretory granules in β-cells. In predisposed, but disease-free individuals, detection of multiple islet cell autoantibodies is a strong predictor for subsequent development of T1D.

### C. Prognostic Indicators

Methods for assessing the signs, symptoms, or complications of T1D are known in the art. Once the diagnosis of diabetes is made, an important goal of therapy is to maintain the average glucose as near the normal range as possible without causing unacceptable amounts of hypoglycemia. The goal for most patients with T1D is to maintain an HbA1c level <7.0% (estimated average glucose of <154 mg/dL). In addition to the HbA1c test, other exemplary methods for assaying the signs, symptoms, or complications of T1D include, but are not limited to, the fasting plasma glucose (FPG) test, the oral glucose tolerance test (OGTT), the random blood glucose test, the C-peptide test, and tests to monitor the levels of T1D-associated autoantibodies.

### D. Prophylactic and Therapeutic Methods

The following discussion is presented by way of example only, and is not intended to be limiting.

One aspect of the present technology provides a method for preventing or delaying the onset of T1D or symptoms of T1D (such as, e.g., hyperglycemia, elevated serum autoantibodies associated with T1D, elevated serum antibodies against exogenous insulin, reduced C-peptide levels) in a subject predisposed to the development of or at risk of having T1D (e.g., first-degree relatives of patients with T1D, where the relatives have been determined to be genetically predisposed to the development of T1D).

Subjects at risk for T1D can be identified by, e.g., any one or a combination of diagnostic or prognostic assays known in the art. In prophylactic applications, insulin-related peptides of the present technology are administered to a subject susceptible to, or otherwise at risk of T1D in an amount sufficient to eliminate or reduce the risk, or delay the onset of the disease, including biochemical and/or behavioral symptoms of the disease, its complications and intermediate pathological phenotypes presenting during development of the disease. Administration of a prophylactic insulin-related peptide can occur prior to the manifestation of symptoms characteristic of the disease, such that the disease is prevented, or alternatively, delayed in its progression.

Subjects at risk for T1D or hyperglycemia include, but are not limited to, subjects who are genetically pre-disposed to T1D, or who are related to a diabetic individual (usually a first-degree relative) or identified to have high-risk HLA genotypes (e.g., the DR3/4-DQ2/8 genotype). Screening for serologic markers including insulin autoantibodies (IAA) and serum autoantibodies associated with islet beta cells (ICA): IA-2A, IA-2β, IAA, GAD-and ZnT8 can also identify individuals at high risk for developing T1D. Assessing C-peptide levels is a widely-used measure of pancreatic β cell function and can also be used to assess an individual's risk for the development of T1D.

Another aspect of the present technology includes methods of treating T1D in a subject diagnosed as having, suspected of having, or at risk of having T1D. In therapeutic applications, compositions comprising insulin-related peptides of the present technology are administered to a subject suspected of, or already suffering from the disease (such as, e.g., subjects exhibiting hyperglycemia, elevated serum autoantibodies associated with T1D, elevated serum antibodies against exogenous insulin, reduced C-peptide levels) in an amount sufficient to cure, or at least partially arrest and delay the onset of, the symptoms of the disease, including its complications. Maintenance of pancreatic beta cell function in treated patients will be indicated by curing or delaying the onset of T1D symptoms, such as reduced C-peptide levels.

In certain embodiments, T1D subjects treated with the sublingual formulations of the insulin-related peptides of the present technology will show normalization of blood glucose levels, T1D-associated autoantibodies, antibodies against exogenous insulin, and/or C-peptide levels by at least 5%, at least 10%, at least 50%, at least 75%, or at least 90% compared to untreated T1D subjects. In certain embodiments, T1D subjects treated with the sublingual formulations of the insulin-related peptides of the present technology will show blood glucose levels, T1D-associated autoantibodies, exogenous insulin associated antibodies and/or C-peptide levels that are similar to that observed in a normal control subject.

### E. Modes of Administration, Pharmaceutical Compositions, and Effective Dosages

In vivo methods typically include the administration of an agent such as those described herein, to a mammal such as a human. When used in vivo for therapy, an agent of the present technology is administered to a mammal in an amount effective in obtaining the desired result or treating the mammal. The dose and dosage regimen will depend upon the degree of the disease in the subject, the characteristics of the particular insulin-related peptide used (e.g., its therapeutic index, duration of action, etc.), the subject, and the subject's history.

An effective amount of an insulin-related peptide of the present technology may be determined during pre-clinical trials and clinical trials by methods familiar to physicians and clinicians. An effective amount of an insulin-related peptide useful in the methods may be administered to a mammal in need thereof by any number of well-known methods for administering pharmaceutical compounds. In particular embodiments, the insulin-related peptides of the present technology are formulated for sublingual administration.

The insulin-related peptides described herein can be incorporated into pharmaceutical compositions for administration, singly or in combination, to a subject for the treatment or prevention of T1D. Such compositions may include the insulin-related peptide and a pharmaceutically acceptable carrier. As used herein, the term “pharmaceutically acceptable carrier” includes a buffer, glycerin, saline, solvents, dispersion media, coatings, antibacterial and antifungal agents, isotonic and absorption delaying agents, and the like, compatible with pharmaceutical administration. Supplementary active compounds can also be incorporated into the compositions.

For the convenience of the patient or treating physician, the dosing formulations can be provided in a kit containing all necessary equipment (e.g., vials of drug, vials of diluent, etc.) for a treatment course.

Sublingual compositions generally include an inert diluent or an edible carrier. For the purpose of sublingual therapeutic administration, the insulin-related peptide can be incorporated with an aqueous pharmaceutically acceptable carrier or excipient (e.g., glycerin) and used in the form of tablets, troches, or capsules. In some embodiments, the aqueous pharmaceutically acceptable carrier comprises at least about 30 vol. % glycerin, at least about 31 vol. % glycerin, at least about 32 vol. % glycerin, at least about 33 vol. % glycerin, at least about 34 vol. % glycerin, at least about 35 vol. % glycerin, at least about 36 vol. % glycerin, at least about 37 vol. % glycerin, at least about 38 vol. % glycerin, at least about 39 vol. % glycerin, at least about 40 vol. % glycerin, at least about 41 vol. % glycerin, at least about 42 vol. % glycerin, at least about 43 vol. % glycerin, at least about 44 vol. % glycerin, at least about 45 vol. % glycerin, at least about 46 vol. % glycerin, at least about 47 vol. % glycerin, at least about 48 vol. % glycerin, at least about 49 vol. % glycerin, at least about 50 vol. % glycerin, at least about 51 vol. % glycerin, at least about 52 vol. % glycerin, at least about 53 vol. % glycerin, at least about 54 vol. % glycerin, at least about 55 vol. % glycerin, or at least about 60 vol. % glycerin. In some embodiments, the aqueous pharmaceutically acceptable carrier comprises at least about 30-70 vol. % glycerin, at least about 35-65 vol. % glycerin, at least about 40-60 vol. % glycerin, at least about 45-60 vol. % glycerin, at least about 50-60 vol. % glycerin, or at least about 50-55 vol. % glycerin. In some embodiments, the aqueous pharmaceutically acceptable carrier further comprises phosphate buffered saline and about 40 to 60 vol. % glycerin. In some embodiments, the aqueous pharmaceutically acceptable carrier further comprise a buffer. The pH can be adjusted with acids or bases, such as hydrochloric acid or sodium hydroxide. Pharmaceutically compatible binding agents and/or adjuvant materials can be included as part of the composition.

Dosage, toxicity, and therapeutic efficacy can be determined by standard pharmaceutical procedures in cell cultures or experimental animals, e.g., for determining the LD50 (the dose lethal to 50% of the population). The dose ratio between toxic and therapeutic effects is the therapeutic index and it can be expressed as the ratio LD50/ED50. Compounds that exhibit high therapeutic indices are advantageous.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods of the present technology, the therapeutically effective dose can be estimated initially from cell culture assays and/or animal studies. Such information can be used to determine useful doses in humans accurately. Levels in plasma may be measured, for example, by high performance liquid chromatography. The dose and dosage regimen will depend upon the degree of the disease in the subject, the characteristics of the particular insulin-related peptide used (e.g., its therapeutic index, duration of action, etc.), the subject, and the subject's history.

Typically, an effective amount of the insulin-related peptides, sufficient for achieving a therapeutic or prophylactic effect, ranges from about 0.000001 mg per kilogram body weight per day to about 10,000 mg per kilogram body weight per day. Suitable, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of peptides ranges from 0.001-10,000 micrograms per kilogram body weight. In one embodiment, insulin-related peptide concentrations in a carrier range from 0.2 to 5000 micrograms per delivered milliliter. In some embodiments, an effective amount of insulin-related peptides sufficient for achieving a therapeutic or prophylactic effect, is measured in units of insulin. For example, dosages can range from 0.5 to 1 unit of insulin/kg body weight/day. An exemplary treatment regimen entails sublingual administration of the insulin-related peptide at least once a day, at least five days a week, for at least 7 weeks. In some embodiments, treatment entails sublingual administration at least once daily for at least 7 weeks. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regimen.

The skilled artisan will appreciate that certain factors may influence the dosage and timing required to effectively treat a subject, including but not limited to, the severity of the disease, previous treatments, the general health and/or age of the subject, and other diseases present.

### EXAMPLES

The present technology is further illustrated by the following examples, which should not be construed as limiting in any way.

### Materials and Methods

Sublingual formulation. A commercial high dose Humulin® insulin solution (Humulin® R U-500) containing 500 units of insulin per mL is mixed with an additional equal volume (1:1 (vol:vol)) 100% glycerin. Each dose contains 10 μL of the Humulin®-glycerin solution, which contains 2.5 units (approximately 87 micrograms) of insulin in a solution having a final concentration of ˜52 vol. % glycerin.

NOD mice. Non-obese diabetic (NOD) mice, as described by Makino (Adv. Immunol. 51:285-322 (1992)), are used in the studies described herein. NOD mice provide a widely accepted animal model for the spontaneous development of Type 1 diabetes. NOD mice develop insulitis as a result of leukocyte infiltration into the pancreatic islet, which in turn leads to the destruction of pancreatic islets and a Type 1 diabetic phenotype.

Serum C-peptide assay. The mouse C-peptide ELISA (ALPCO), which quantifies C-peptide protein products of mouse I and mouse II proinsulin genes, was used. Briefly, mice are fasted overnight and blood is collected by inserting a needle into the submandibular vein and collecting ˜0.2 mL of blood. The blood is centrifuged for 10 minutes at 3000×g in a refrigerated centrifuge, and serum is collected and stored −80° C. The ALPCO C-peptide ELISA is a commercially available FDA Registered For In Vitro Diagnostic Use tool for the quantification of human C-peptide in serum and plasma samples.

Autoantibody titer assay. To detect anti-insulin antibodies 96-well ELISA plates were coated with 1 μg/well of Humulin® overnight at 4° C. and were blocked with 1% BSA in PBS. Sera were divided into two equal aliquots that were incubated with or without 10 μg/ml Humulin® on ice for 1 h. The sera were then added to Humulin® coated plates for incubation overnight at 4° C. After extensive washes, the plates were incubated with HRP-conjugated goat anti-mouse IgG The assay is described in Wan et al., J Exp Med. 2016 May 30; 213(6): 967-978.

### Example 1: Use of Insulin-Related Peptides in Delaying the Onset of Hyperglycemia in a Mouse Model of Type 1 Diabetes

This Example demonstrates the use of a sublingual formulation of insulin-related peptides of the present technology in methods for delaying the onset of hyperglycemia in a mouse model of Type 1 diabetes.

### Methods

Five-week old female NOD mice were randomly assigned to three groups: (1) control group (1:1 glycerin/phosphate buffered saline (PBS)); (2) insulin-related peptide treatment (Humulin®) started at six weeks of age; or (3) insulin-related peptide treatment (Humulin®) started at ten weeks of age. Mice in the treatment groups (2) and (3) were sublingually administered 2.5 units/87 μg of Humulin® R insulin (10 μL of solution) twice per day five times per day from from age 5 weeks 5 until 8 weeks of age and then once per day, five days per week up to 30 weeks of age. Blood glucose measurements were taken once per week up to 20 weeks of age, and then twice per week thereafter. Mice were classified as diabetic after three consecutive blood glucose readings above 300 mg/dL (hyperglycemic).

### Results

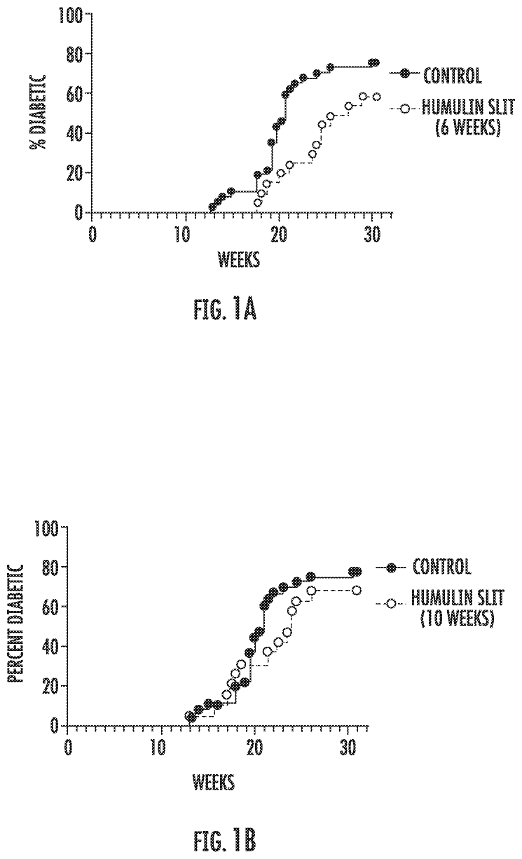

As shown in FIG. 1A, treatment with sublingual Humulin® insulin significantly reduced both the incidence (% T1D Onset) of Type 1 diabetes and onset time (weeks) of Type 1 diabetes in treatment group 2 (i.e., mice treated with Humulin® starting at six (6) weeks of age) as compared to the control group. FIG. 1B, shows the results from treatment group 3 (i.e., mice treated with Humulin® starting at ten (10) weeks of age) as compared to the control group. Table 3 provides the statistics associated with the survival curves shown in FIGS. 1A and 1B.

TABLE 3
Comparison of Survival Curves

| | FIG. 1A | FIG. 1B |
|---|---|---|
| Log-rank (Mantel-Cox) test | | |
| Chi square | 3.98 | 0.812 |
| Df | 1 | 1 |
| P value | 0.0460 | 0.367 |
| P value summary | * | ns |
| Are the survival curves significantly | Yes | No |
| different? | | |
| Gehan-Breslow-Wilcoxon test | | |
| Chi square | 5.45 | 0.725 |
| Df | 1 | 1 |
| P value | 0.020 | 0.389 |
| P value summary | * | ns |
| Are the survival curves significantly | Yes | No |
| different? | | |
| Median Survival (i.e., time to Type 1 diabetes onset) | | |
| Control | 21 weeks | 21 weeks |
| 6/10 wk Humulin initiation | 27 weeks | 24 weeks |
| Ratio (and its reciprocal) | 0.778 (1.29) | 0.8750 (1.14) |
| 95% CI of ratio | 0.396 to 1.53 | 0.453 to 1.69 |
| | (0.654 to 2.53) | (0.592 to 2.21) |

These results demonstrate that the sublingual formulations of insulin-related peptides of the present technology, such as Humulin® insulin, can be useful in methods for ameliorating the onset of Type 1 diabetes, where treatment includes delaying the onset of hyperglycemia or decreasing the likelihood of developing Type 1 diabetes in a subject.

### Example 2: Use of Insulin-Related Peptides in Attenuating an Antigenic Response

This Example demonstrates the use of a sublingual formulation of insulin-related peptides of the present technology in methods for attenuating an antigenic response in subjects at risk for or having been diagnosed with Type 1 diabetes. The onset of Type 1 diabetes is preceded and accompanied by the appearance of a number of autoantibodies to a variety of pancreatic islet cell antigens. In genetically predisposed, but disease-free, individuals (e.g., first-degree relatives of patients with Type 1 diabetes), detection of multiple islet cell autoantibodies is a strong predictor for subsequent development of Type I diabetes. These autoantibodies include, but are not limited to, islet cell antibodies (ICA, against cytoplasmic proteins in the beta cell), antibodies to glutamic acid decarboxylase (GAD-65), insulin autoantibodies (IAA), and autoantibodies to tyrosine phosphatases IA-2A and IA-2β, and ZnT8.

### Methods

Five-week old female NOD mice were randomly assigned to two groups: (1) control group (50% PBS/glycerin); and (2) insulin-related peptide treatment (Humulin® insulin) started at six weeks of age. Mice in the treatment group (2) were sublingually administered 87 μg of Humulin® in 50% glycerin solution twice per day, five days per week, up to 30 weeks of age. Serum samples were collected from control and Humulin® SLIT treated NOD mice at 14 weeks of age and assessed for levels of anti-insulin antibody titer (i.e., after sublingual administration of Humulin® for 8 weeks) and stored at −80 C until tested for anti-insulin antibodies in the ELISA described above. The level of anti-insulin antibodies in the samples was determined by ELISA using the assay described in Wan et al., J Exp Med. 2016 May 30; 213(6): 967-978 and the results are shown in FIG. 2.

### Results

As shown in FIG. 2, treatment with sublingual Humulin® significantly reduced the development of anti-insulin antibodies in treatment group 2 (i.e., mice treated with 87 μg of Humulin® starting at six (6) weeks of age as compared to the control group, as determined from serum samples obtained from the NOD mice at 14 weeks of age. These results demonstrate that the sublingual formulations of insulin-related peptides of the present technology, such as Humulin® insulin or an insulin variant having one or more conservative amino acid substitutions, are useful in methods for suppressing an antigenic response to Type 1 diabetes related-antigens as compared to untreated controls.

### Example 3: Use of Insulin-Related Peptides in Delaying the Onset of Hyperglycemia in NOD Mice

This Example demonstrates the use of a sublingual formulation of insulin-related peptides of the present technology in methods for delaying the onset of hyperglycemia in a mouse model of Type 1 diabetes.

### Methods

Female NOD mice were randomly assigned to two groups: (1) control group (50% glycerin/phosphate buffered saline (PBS)); or (2) insulin-related peptide treatment (Humulin® in 50% glycerin solution; Humulin® sublingual immunotherapy (“Humulin SLIT”)) started at five weeks of age. Mice in the treatment group (2) were sublingually administered 87 μg of Humulin® once per day, five days per week starting at five weeks of age up to 30 weeks of age. Blood glucose measurements were taken once per week up to 13 weeks of age, and then twice per week thereafter. Mice were classified as diabetic after three consecutive blood glucose readings above 300 mg/dL (hyperglycemic).

### Results

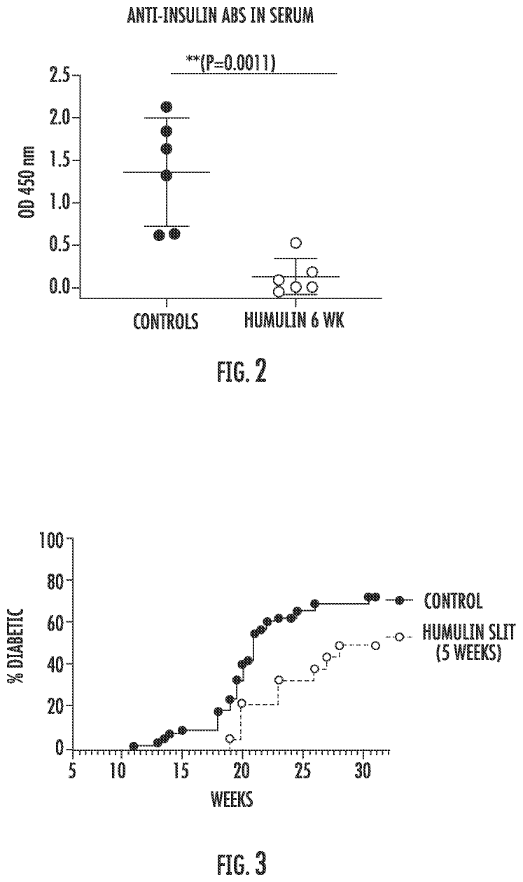

As shown in FIG. 3, treatment with sublingual Humulin® reduced both the incidence (% diabetic) of Type 1 diabetes and onset time (weeks) of Type 1 diabetes in treatment group 2 (i.e., mice treated with Humulin® insulin starting at five (5) weeks of age) as compared to the control group. Table 4 provides the statistics associated with the survival curves shown in FIG. 3.

TABLE 4
Comparison of Survival Curves

| | | |
|---|---|---|
| Log-rank (Mantel-Cox) test | | |
| Chi square | 4.531 | |
| df | 1 | |
| P value | 0.0333 | |
| P value summary | * | |
| Are the survival curves sig different? | Yes | |
| Gehan-Breslow-Wilcoxon test | | |
| Chi square | 5.499 | |
| df | 1 | |
| P value | 0.0190 | |
| P value summary | * | |
| Are the survival curves sig different? | Yes | |
| Median survival | | |
| Control | 21.00 | |
| 5 wk Humulin | 29.50 | |
| Ratio (and its reciprocal) | 0.7119 | 1.405 |
| 95% CI of ratio | 0.3436 to 1.475 | 0.6780 to 2.911 |

These results demonstrate that the sublingual formulations of insulin-related peptides of the present technology, such as Humulin® insulin, are useful in methods for treating Type 1 diabetes, where treatment includes delaying the onset of hyperglycemia or decreasing the likelihood of developing Type 1 diabetes in a subject.

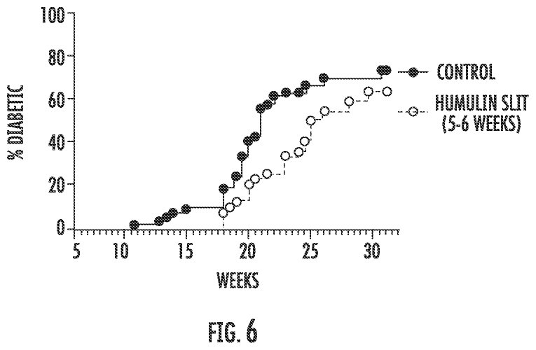

The results of the treatment of NOD mice sublingually with 87 μg of Humulin® insulin once per day starting at 5 weeks of age in this Example 3 (see FIG. 3) were combined for analytical purposes with the results of the treatment of NOD mice sublingually with 87 μg of Humulin® insulin once per day starting at 6 weeks of age described in Example 1 above (see FIG. 1A). The analysis of the combined results is summarized in Table 5, which provides the statistics associated with the survival curves shown in FIG. 6. The analysis of the combined results provides additional confirmation that the sublingual formulations of insulin-related peptides, such as Humulin® insulin, can be useful in methods for ameliorating the onset of Type 1 diabetes, where treatment includes delaying the onset of hyperglycemia or decreasing the likelihood of developing Type 1 diabetes in a subject.

TABLE 5
Comparison of Survival Curves

| | | |
|---|---|---|
| Log-rank (Mantel-Cox) test | | |
| Chi square | 4.683 | |
| df | 1 | |
| P value | 0.0305 | |
| P value summary | * | |
| Are the survival curves sig different? | Yes | |
| Gehan-Breslow-Wilcoxon test | | |
| Chi square | 7.010 | |
| df | 1 | |
| P value | 0.0081 | |
| P value summary | ** | |
| Are the survival curves sig different? | Yes | |
| Median survival | | |
| Control | 21.00 | |
| 5 wk Humulin | 26.00 | |
| Ratio (and its reciprocal) | 0.8077 | 1.238 |
| 95% CI of ratio | 0.4688 to 1.392 | 0.7186 to 2.133 |

### Example 4: Use of Insulin-Related Peptides in Attenuating an Antigenic Response

This Example demonstrates the use of a sublingual formulation of insulin-related peptides of the present technology in methods for attenuating an antigenic response in subjects at risk for or having been diagnosed with Type 1 diabetes. The onset of Type 1 diabetes is preceded and accompanied by the appearance of a number of autoantibodies to a variety of pancreatic islet cell antigens. In genetically predisposed, but disease-free, individuals (e.g., first-degree relatives of patients with Type 1 diabetes), detection of multiple islet cell autoantibodies is a strong predictor for subsequent development of Type I diabetes. These autoantibodies include, but are not limited to, islet cell antibodies (ICA, against cytoplasmic proteins in the beta cell), antibodies to glutamic acid decarboxylase (GAD-65), insulin autoantibodies (IAA), and autoantibodies to tyrosine phosphatases IA-2A and IA-2β, and ZnT8.

### Methods

Female NOD mice were randomly assigned and treated in two groups as described in Example 3. Serum samples were collected from control and Humulin® SLIT treated NOD mice at 19 weeks of age and assessed for levels of anti-insulin antibody titer (i.e., after sublingual administration of Humulin® for 14 weeks). The level of anti-insulin antibodies in the samples was determined by ELISA using the assay described in Wan et al., J. Exp. Med. 2016 May 30; 213(6): 967-978 and the results are shown in FIG. 4.

### Results

As shown in FIG. 4, treatment with sublingual Humulin® significantly reduced the development of anti-insulin antibodies in treatment group 2 (i.e., mice treated with 87 μg of Humulin® starting at five (5) weeks of age) as compared to the control group, as determined from serum samples obtained from the NOD mice at 19 weeks of age. These results demonstrate that the sublingual formulations of insulin-related peptides of the present technology, such as Humulin® or a variant of an insulin having one or more conservative amino acid substitutions, are useful in methods for suppressing an antigenic response to Type 1 diabetes related-antigens as compared to untreated controls.

### Example 5: Use of Insulin-Related Peptides in Conserving Serum C-Peptide Levels

This Example demonstrates the use of a sublingual formulation of insulin-related peptides of the present technology in methods for conserving serum C-peptide levels in a mouse model of Type 1 diabetes. C-peptide is the portion of proinsulin joining the alpha and beta insulin chains that is cleaved out prior to co-secretion with insulin from pancreatic beta cells. Produced in equimolar amounts to endogenous insulin, the 31-amino acid C-peptide is not a product of therapeutically administered exogenous insulin and has been widely used as a measure of insulin secretion (or pancreatic beta cell function). (See, e.g., Leighton et al., Diabetes Ther. 2017 June; 8(3): 475-487) and Wan et al., J Exp Med. 2016 May 30; 213(6): 967-978.

### Methods

Female NOD mice were randomly assigned to two groups according to Example 3. C-peptide levels in serum were determined by ALPCO C-peptide ELISA from serum samples collected at 6 and 19 weeks of age in mice that were fasted overnight.

### Results

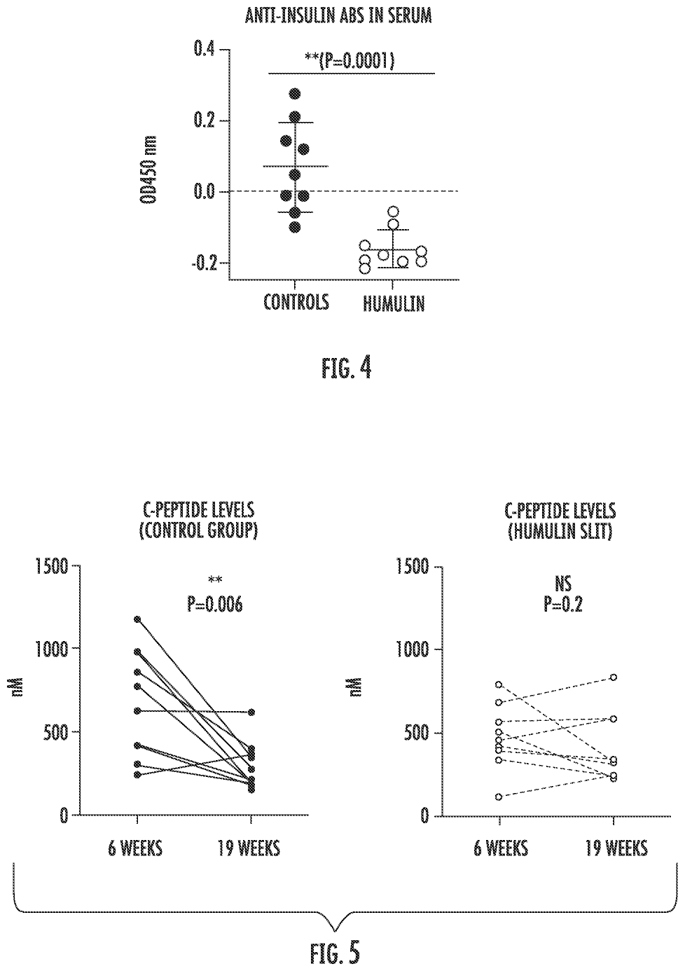

Overall, as shown in FIG. 5, on average NOD mice that received treatment with sublingual Humulin® displayed either a maintenance or enhancement of serum C-peptide levels as compared to untreated control NOD mice, which, overall, displayed significant reductions in serum C-peptide levels. The results demonstrate that the sublingual formulations of insulin-related peptides of the present technology, such as Humulin® insulin or an insulin variant having one or more conservative amino acid substitutions, can be useful in methods for the treatment of Type 1 diabetes in a subject, including those with biological markers or history indicating a predisposition to the development of Type 1 diabetes.

### Example 6: Use of Insulin-Related Peptides in Attenuating an Antigenic Response

This Example demonstrates the use of a sublingual formulation of insulin-related peptides of the present technology in methods for attenuating an antigenic response in subjects at risk for or having been diagnosed with Type 1 diabetes. The onset of Type 1 diabetes is preceded and accompanied by the appearance of a number of autoantibodies to a variety of pancreatic islet cell antigens. In genetically predisposed, but disease-free, individuals (e.g., first-degree relatives of patients with Type 1 diabetes), detection of multiple islet cell autoantibodies is a strong predictor for subsequent development of Type I diabetes. These autoantibodies include, but are not limited to, insulin autoantibodies (IAA).

### Methods

Female NOD mice were randomly assigned to two groups: (1) control group (50% glycerin/phosphate buffered saline (PBS)); or (2) insulin-related peptide treatment (Humulin® insulin, preproinsulin synthetic peptide, and insulin beta chain 9-23 synthetic peptide in ˜50% glycerin solution; Humulin®+Peptides sublingual immunotherapy (“Peptides SLIT”)) started at five (5) weeks of age. Mice in the treatment group (2) were sublingually administered 10 μL of a composition comprising 52 μg of Humulin®, 10 μg of preproinsulin synthetic peptide, and 10 μg insulin beta chain 9-23 synthetic peptide once per day, five days per week starting at five (5) weeks of age up to 30 weeks of age. Blood glucose measurements were taken once per week up to 13 weeks of age, and then twice per week thereafter. Mice were classified as diabetic after three consecutive blood glucose readings above 300 mg/dL (hyperglycemic).

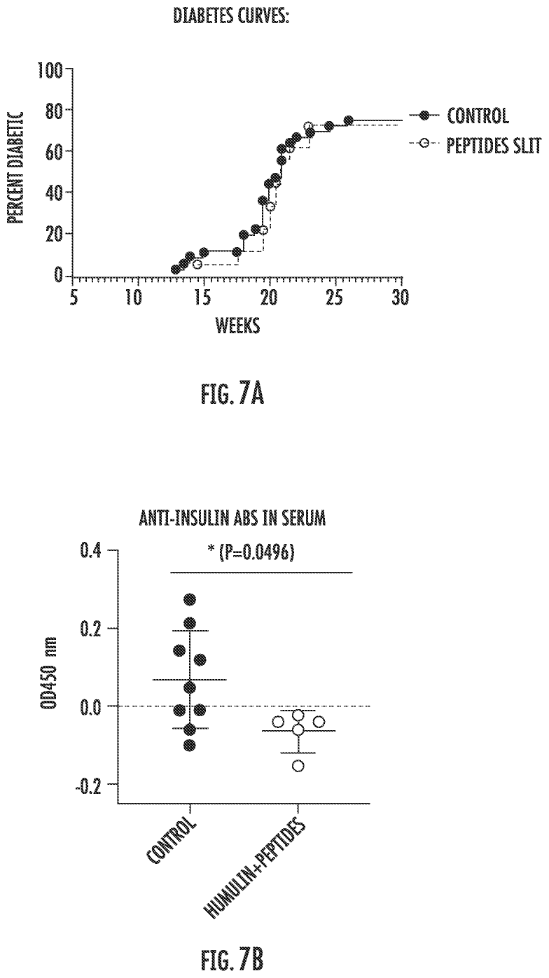

Serum samples were collected from control and Humulin®+Peptides SLIT treated NOD mice at 14 weeks of age and assessed for levels of anti-insulin antibody titer (i.e., after sublingual administration of Humulin® for 9 weeks). The level of anti-insulin antibodies in the samples was determined by ELISA using the assay described in Wan et al., J Exp Med. 2016 May 30; 213(6): 967-978 and the results are shown in FIG. 7B.

### Results

Although treatment with Humulin+Peptides SLIT (Peptides SLIT) did not significantly affect the incidence of Type 1 diabetes or onset time of Type 1 diabetes in the treatment group as compared to the control group (FIG. 7A), the data shown in FIG. 7B demonstrates that treatment with sublingual Humulin®, preproinsulin synthetic peptide, and insulin beta chain 9-23 synthetic peptide significantly reduced the development of anti-insulin antibodies in treatment group 2 (i.e., mice treated with 52 μg Humulin®, 10 μg of preproinsulin synthetic peptide, and 10 μg insulin beta chain 9-23 synthetic peptide starting at six (6) weeks of age) as compared to the control group, as determined from serum samples obtained from the NOD mice at 14 weeks of age. These results demonstrate that the sublingual formulations of insulin-related peptides of the present technology, such as compositions comprising Humulin®, preproinsulin synthetic peptide, and insulin beta chain 9-23 synthetic peptide, or a variant of an insulin having one or more conservative amino acid substitutions, are useful in methods for suppressing an antigenic response to Type 1 diabetes related-antigens as compared to untreated controls.

### Example 7: Use of Insulin-Related Peptides in Delaying the Onset of Hyperglycemia in Humans

This Example demonstrates the use of a sublingual formulation of insulin-related peptides of the present technology in methods for treating Type 1 diabetes in disease-free, individuals predisposed to the development of Type 1 diabetes (e.g., first-degree relatives of patients with Type 1 diabetes, where the relatives have been determined to be genetically predisposed to the development of Type 1 diabetes).

### Methods

Subjects determined to be predisposed to the development of Type 1 diabetes receive daily, sublingual administrations of an insulin-related peptide of the present technology. Dosages will range between 0.1 mg/kg to 50 mg/kg. Subjects will be evaluated weekly for the presence and/or severity of signs and symptoms associated with Type 1 diabetes, including, but not limited to, e.g., hyperglycemia, hypoinsulinemia, serum C-peptide levels, A1C levels, or presence of autoantibodies. Treatments may be maintained indefinitely or until such time as one or more signs or symptoms of Type 1 diabetes develop.

### Results

It is predicted that subjects predisposed to the development of Type 1 diabetes receiving sublingually administered therapeutically effective amounts of insulin-related peptides of the present technology will display delayed and/or reduced severity or elimination of the signs or symptoms associated with the development of Type 1 diabetes. These results will show that sublingual formulations of insulin-related peptides of the present technology, such as Humulin® or a biologically active fragment thereof or a variant of either of these having one or more conservative amino acid substitutions, are useful in the treatment of Type 1 diabetes in a subject in need thereof and in particular, in delaying the onset of hyperglycemia and/or decreasing the likelihood of developing Type 1 diabetes in the subject.

### ILLUSTRATIVE EMBODIMENTS

In one aspect, a method for delaying the onset of reduced serum C-peptide levels in a mammal is provided. The method comprises sublingually administering an effective amount of an insulin-related peptide to the mammal.

In another aspect, a method for conserving pancreatic beta cell function in a mammal is provided. The method comprises sublingually administering an insulin-related peptide to the mammal in an amount effective to at least delay a reduction in serum C-peptide levels in the mammal.

In another aspect, a method for delaying the onset of decreased pancreatic beta cell function in a mammal is provided. The method comprises sublingually administering an insulin-related peptide to the mammal in an amount effective to conserve serum C-peptide levels in the mammal.

In another aspect, a method for attenuating an antigenic response in a mammal to at least one Type 1 diabetes related-antigen is provided. The method comprises sublingually administering an insulin-related peptide to the mammal in an amount of effective to inhibit development of antibodies to at least one Type 1 diabetes related-antigen. The Type 1 diabetes related-antigen typically comprises one or more of insulin, glutamic acid decarboxylase 65 (GAD65), insulinoma-associated protein 2 (IA-2), zinc transporter-8 (ZnT8), and islet amyloid polypeptide (IAPP). Very often, the method comprises attenuating the antigenic response in the mammal to an insulin and, optionally, one or more other Type 1 diabetes related-antigens. The method may result in comprises inhibiting development of anti-insulin antibodies (IA) in the mammal after sublingual administration of the insulin-related peptide as compared to a control mammalian subject.

In any of the methods described in paragraphs [0086] to [0089], the insulin-related peptide may be administered at least once a day, at least five days a week, for at least 7 weeks. In some instances, the insulin-related peptide may be administered at least twice a day, at least five days a week, for at least 7 weeks. In some instances, the insulin-related peptide may be administered at least once daily for at least 7 weeks.

In any of the methods described in paragraphs [0086] to [0090], the insulin-related peptide may include a first amino acid sequence comprising an insulin beta chain 7-26 peptide sequence (SEQ ID NO: 9) or a variant thereof having one or more amino acid substitutions; and a second amino acid sequence comprising an insulin alpha chain 6-20 peptide sequence (SEQ ID NO: 4) or a variant thereof having one or more amino acid substitutions. For example, the insulin-related peptide comprises an insulin, such as a human insulin. In some instances, the insulin-related peptide is a recombinant human insulin-related peptide.

In any of the methods described in paragraphs [0086] to [0091], mammalian subject may be predisposed to the development of Type 1 diabetes. For example, the mammalian subject may be an NOD mouse. In other instances, the mammalian subject may be a human subject, e.g., a human subject at risk of the development of Type 1 diabetes, such as a first-degree relative of a patient with type 1 diabetes and/or a human subject genetically predisposed to developing type 1 diabetes. Examples of such genetically predisposed human subjects include human subjects having a high-risk HLA genotype (e.g., a DR3/4-DQ2/8 genotype).

In any of the methods described in paragraphs [0086] to [0092], the method may include sublingually administering a composition comprising the effective amount of the insulin-related peptide; and an aqueous pharmaceutically acceptable carrier, which comprises at least about 30 vol. % glycerin. The aqueous pharmaceutically acceptable carrier may also include a buffer, such as a phosphate buffer. Typically, the aqueous pharmaceutically acceptable carrier includes about 40 to 60 vol. % glycerin. Quite often, the composition may also include a preservative (e.g., meta-cresol) and/or a zinc source (e.g., zinc oxide). The compositions administered in these methods suitably includes at least about 2 micrograms insulin-related peptide per μL and, often, at least about 5 micrograms insulin-related peptide per μL of the composition—e.g., about 5 to 10 micrograms insulin-related peptide per μL of the composition. In some instances, it may be suitable to use a composition containing the insulin-related peptide where the aqueous pharmaceutically acceptable carrier comprises phosphate buffered saline and about 40 to 60 vol. % glycerin.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The foregoing description is provided to assist the understanding of the reader. None of the information provided or references cited is admitted to be prior art to the compositions and methods disclosed herein.

All patents, patent applications, provisional applications, and publications referred to or cited herein are incorporated by reference in their entirety, including all figures and tables, to the extent they are not inconsistent with the explicit teachings of this specification.

1. A method for delaying the onset of reduced serum C-peptide levels in a mammal, conserving pancreatic beta cell function in a mammal, or delaying the onset of decreased pancreatic beta cell function in a mammal, comprising:
sublingually administering an effective amount of an insulin-related peptide to the mammal.

2.-7. (canceled)

8. The method of claim 1, wherein the insulin-related peptide is administered at least once a day, at least five days a week, for at least 7 weeks.

9. The method of claim 8, wherein the insulin-related peptide is administered at least once daily for at least 7 weeks.

10. The method of claim 1, wherein the insulin-related peptide comprises a first amino acid sequence comprising an insulin beta chain 7-26 peptide sequence (SEQ ID NO: 9) or a variant thereof having one or more amino acid substitutions; and a second amino acid sequence comprising an insulin alpha chain 6-20 peptide sequence (SEQ ID NO: 4) or a variant thereof having one or more amino acid substitutions.

11. The method of claim 1, wherein the insulin-related peptide comprises an insulin.

12. The method of claim 1, wherein the insulin-related peptide comprises human insulin.

13. The method of claim 1, wherein the insulin-related peptide is a recombinant human insulin-related peptide.

14. The method of claim 1, wherein the mammal is a human.

15. The method of claim 1, wherein the mammal is predisposed to the development of Type 1 diabetes.

16. (canceled)

17. The method of claim 15, wherein the mammal is a human subject.

18. The method of claim 17, wherein the human subject is a first-degree relative of a patient with type 1 diabetes.

19. The method of claim 17, wherein the human subject is genetically predisposed to developing type 1 diabetes.

20. The method of claim 19, wherein the human subject has a high-risk HLA genotype (e.g., a DR3/4-DQ2/8 genotype).

21. The method of claim 1, wherein the method comprises sublingually administering a composition comprising:
(i) the effective amount of the insulin-related peptide; and
(ii) an aqueous pharmaceutically acceptable carrier, which comprises at least about 30 vol. % glycerin.

22. The method of claim 21, wherein the aqueous pharmaceutically acceptable carrier further comprises a buffer.

23. The method of claim 21, wherein the aqueous pharmaceutically acceptable carrier comprises about 40 to 60 vol. % glycerin.

24. The method of claim 21, wherein the composition further comprises a preservative and/or a zinc source.

25. The method of claim 24, wherein the composition comprises meta-cresol and zinc oxide.

26. The method of claim 21, wherein the composition comprises at least about 5 micrograms insulin-related peptide per μL of the composition.

27. The method of claim 21, wherein the aqueous pharmaceutically acceptable carrier comprises phosphate buffered saline and about 40 to 60 vol. % glycerin.


SEQUENCE LISTING (24 sequences)

SEQ ID NO: 1 - protein, 30 aa, Artificial Sequence
  Description of Artificial Sequence Synthetic polypeptide
FVNQHLCGSHLVEALYLVCGERGFFYTPKT

SEQ ID NO: 2 - protein, 21 aa, Artificial Sequence
  Description of Artificial Sequence Synthetic peptide
GIVEQCCTSICSLYQLENYCN

SEQ ID NO: 3 - protein, 21 aa, Artificial Sequence
  Description of Artificial Sequence Synthetic peptide
LCGSHLVEALYLVCGERGFFY

SEQ ID NO: 4 - protein, 15 aa, Artificial Sequence
  Description of Artificial Sequence Synthetic peptide
CCTSICSLYQLENYC

SEQ ID NO: 5 - protein, 20 aa, Artificial Sequence
  Description of Artificial Sequence Synthetic peptide
GIVEQCCTSICSLYQLENYC

SEQ ID NO: 6 - protein, 17 aa, Artificial Sequence
  Description of Artificial Sequence Synthetic peptide
EQCCTSICSLYQLENYC

SEQ ID NO: 7 - protein, 24 aa, Artificial Sequence
  Description of Artificial Sequence Synthetic peptide
NQHLCGSHLVEALYLVCGERGFFY

SEQ ID NO: 8 - protein, 24 aa, Artificial Sequence
  Description of Artificial Sequence Synthetic peptide
QHLCGSHLVEALYLVCGERGFFYT

SEQ ID NO: 9 - protein, 20 aa, Artificial Sequence
  Description of Artificial Sequence Synthetic peptide
CGSHLVEALYLVCGERGFFY

SEQ ID NO: 10 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 11 - protein, 86 aa, Unknown; identical to SEQ ID NO: 10
  Description of Unknown Great ape sequence

SEQ ID NO: 12 - protein, 86 aa, Macaca fascicularis
FVNQHLCGSHLVEALYLVCGERGFFYTPKTRREAEDPQVGQVELGGGPGAGSLQPLALEGSLQKRGIVEQCCTSICSLYQLENYCN

SEQ ID NO: 13 - protein, 86 aa, Oryctolagus sp.
FVNQHLCGSHLVEALYLVCGERGFFYTPKSRREVEELQVGQAELGGGPGAGGLQPSALELALQKRGIVEQCCTSICSLYQLENYCN

SEQ ID NO: 14 - protein, 86 aa, Canis sp.
FVNQHLCGSHLVEALYLVCGERGFFYTPKARREVEDLQVRDVELAGAPGEGGLQPLALEGALQKRGIVEQCCTSICSLYQLENYCN

SEQ ID NO: 15 - protein, 86 aa, Equus sp.
  MOD_RES (31)..(32): Any amino acid
  MOD_RES (64)..(65): Any amino acid
FVNQHLCGSHLVEALYLVCGERGFFYTPKAXXEAEDPQVGEVELGGGPGLGGLQPLALAGPQQXXGIVEQCCTGICSLYQLENYCN

SEQ ID NO: 16 - protein, 84 aa, Sus sp.
FVNQHLCGSHLVEALYLVCGERGFFYTPKARREAENPQAGAVELGGGLGGLQALALEGPPQKRGIVEQCCTSICSLYQLENYCN

SEQ ID NO: 17 - protein, 86 aa, Unknown
  Description of Unknown Hamster sequence
FVNQHLCGSHLVEALYLVCGERGFFYTPKSRRGVEDPQVAQLELGGGPGADDLQTLALEVAQQKRGIVDQCCTSICSLYQLENYCN

SEQ ID NO: 18 - protein, 86 aa, Rattus sp.
FVKQHLCGSHLVEALYLVCGERGFFYTPMSRREVEDPQVAQLELGGGPGAGDLQTLALEVARQKRGIVDQCCTSICSLYQLENYCN

SEQ ID NO: 19 - protein, 86 aa, Mus sp.
FVKQHLCGSHLVEALYLVCGERGFFYTPMSRREVEDPQVAQLELGGGPGAGDLQTLALEVAQQKRGIVDQCCTSICSLYQLENYCN

SEQ ID NO: 20 - protein, 86 aa, Felis sp.
FVNQHLCGSHLVEALYLVCGERGFFYTPKARREAEDLQGKDAELGEAPGAGGLQPSALEAPLQKRGIVEQCCASVCSLYQLEHYCN

SEQ ID NO: 21 - protein, 86 aa, Rattus sp.
FVKQHLCGPHLVEALYLVCGERGFFYTPKSRREVEDPQVPQLELGGGPEAGDLQTLALEVARQKRGIVDQCCTSICSLYQLENYCN

SEQ ID NO: 22 - protein, 84 aa, Mus sp.
FVKQHLCGPHLVEALYLVCGERGFFYTPKSRREVEDPQVEQLELGGSPGDLQTLALEVARQKRGIVDQCCTSICSLYQLENYCN

SEQ ID NO: 23 - protein, 81 aa, Bos sp.
FVNQHLCGSHLVEALYLVCGERGFFYTPKARREVEGPQVGALELAGGPGAGGLEGPPQKRGIVEQCCASVCSLYQLENYCN

SEQ ID NO: 24 - protein, 81 aa, Ovis sp.
FVNQHLCGSHLVEALYLVCGERGFFYTPKARREVEGPQVGALELAGGPGAGGLEGPPQKRGIVEQCCAGVCSLYQLENYCN